(19) United States
(12) Patent Application Publication
(10) Pub. No.: US 2013/0053896 A1
(43) Pub. Date: Feb. 28, 2013

(54) ADAPTABLE SYSTEMS, METHODS, AND DEVICES FOR PERCUTANEOUSLY IMPLANTING A SPINAL SCREW

(71) Applicant: Jean-Marc VOYADZIS, Washington, DC (US)

(72) Inventor: Jean-Marc VOYADZIS, Washington, DC (US)

(21) Appl. No.: 13/595,188

(22) Filed: Aug. 27, 2012

Related U.S. Application Data
(60) Provisional application No. 61/575,734, filed on Aug. 29, 2011.

(51) Int. Cl. A61B 17/88; A61B 17/86

(52) U.S. Cl. 606/279; 606/305

(57) ABSTRACT

Systems, devices, and methods for percutaneously implanting a spinal screw provide reduced trauma to soft tissues, less blood loss and postoperative pain, less scarring, and faster mobilization compared to open spinal procedures. The devices and methods provide techniques for percutaneous insertion of pedicle screws or other screws without the use of a guide wire. Screw extenders are paired and include extending branches with elongated portions for extending from a proximal end outside a patient to a distal end. The screw extenders include interlocking tabs and other locking mechanisms at the distal ends that connect at the undersurface of a screw head and include a graduated bilateral locking mechanism to ensure a positive connection between the paired extender branches. The screw extenders are used through a working channel firmly held in place by a table-mounted flexible arm. The screw extenders can be used on available pedicle screw systems to convert their insertion to a percutaneous technique.

### CROSS-REFERENCE TO RELATED APPLICATION

This application claims benefit of priority of U.S. Provisional Patent Application Ser. No. 61/575,734 filed on Aug. 26, 2011, the entire disclosure of which is incorporated herein by reference.

### TECHNICAL FIELD

This technology relates to spinal instrumentation systems and spinal surgical treatment. More particularly, the technology relates to systems, devices, and methods of percutaneous screw insertion, that is, the implantation of a fixation device into vertebral bone with minimal invasion into the surrounding body tissue.

### BACKGROUND

The insertion of screws in the spine is performed when patients develop vertebral instability from trauma, degenerative conditions, cancer, and other disease processes. Screw implantation allows for solid fixation into bone and can be incorporated with other instruments or devices to impart immediate spinal stability to facilitate healing.

One technique of screw insertion involves broad surgical exposure of the bony elements of the spine and implantation under direct visualization. This invasive surgery is extensive and requires detachment of and retraction onto the surrounding muscles and ligaments that cause bleeding, muscle injury, and significant postoperative pain. The technique of percutaneous insertion of spinal screws has been developed to decrease tissue dissection, minimize blood loss, and mitigate discomfort after surgery. Percutaneous insertion involves much smaller incisions and screw implantation under fluoroscopic x-ray guidance. Percutaneous screw placement has resulted in faster patient recovery in many circumstances.

Percutaneous screw systems (pedicle or other) often require cannulated instruments with guide wire insertion to direct placement. The use of guide wires adds to operative risk with potential complications such as guide wire breakage in the patient, guide wire insertion onto neural structures or into the abdominal cavity, and spinal fluid leak. Screw insertion with the use of guide wires is technically demanding, requires a greater learning curve, and lengthens surgery time. This technique also requires significant intraoperative x-ray imaging with the potential for harm to the patient, surgeon, and operating room staff. Additionally, the development of special cannulated instruments adds cost to the procedures, which is ultimately passed on to the public. These factors have limited the appeal of percutaneous screw insertion devices to spine surgeons despite the fact that minimally invasive spinal instrumentation has been shown to significantly decrease blood loss, reduce the rate of infection, and diminish postoperative pain with the potential to improve patient outcomes.

### SUMMARY

The systems, devices, and methods for percutaneously implanting a spinal screw in accordance with the claimed invention provide reduced trauma to the bones and soft tissues, less postoperative pain, less scarring, faster mobilization, and reduced mortality from surgical blood loss compared to open or other spinal procedures.

The devices and methods in accordance with the claimed invention provide a novel technique of percutaneous spinal screw insertion. These methods of insertion requires an access needle that once inserted into the pedicle and vertebral body, serves as a guide for the insertion of tubular dilators and ultimately a working channel. This working channel, once fixed in space, enables insertion of preparatory instruments and pedicle screws through it. No guide wire is used, and the aforementioned potential complications are avoided. Less x-ray imaging is required as a result of this. Insertion techniques are performed through the working channel, and instrumentation with minor modifications make these methods appear familiar to the spine surgeon.

According to another aspect of the claimed invention, paired screw extenders are designed to lock under the head of standard pedicle screws to allow for their percutaneous insertion through the aforementioned working channel. These embodiments allow easy conversion of a standard screw insertion set to a percutaneous screw insertion set with reduction of costs and more widespread appeal.

One aspect of the claimed invention includes a method of securing a fixation device into a vertebral bone. The method includes inserting an access needle with a removable handle in the vertebral bone, inserting sequentially larger dilators over the access needle, and inserting a working channel over the sequentially larger dilators. The method further includes securing the working channel to provide stable access to the vertebral bone, removing the dilators and access needle, and attaching a set of screw extenders to the fixation device. The fixation device is then inserted with the attached set of screw extenders into the working channel and the fixation device is secured into the vertebral bone through the working channel without the use of a guide wire.

In one example implementation, the fixation device is a pedicle screw. In one example implementation, a plurality of fixation devices are secured in at least one vertebral bone. Additionally, a support or alignment device, such as a rod is installed to the plurality of fixation devices. The method of securing a fixation device into a vertebral bone where the screw extender is attached to the fixation device can include connecting interlocking tabs on the screw extender at an undersurface of the fixation device. The set of screw extenders can be locked to the fixation device.

The locking mechanism for locking the set of screw extenders to the fixation device can include one or more locking mechanisms including, for example, a tab and slot locking mechanism on the screw extender, a spring-loaded clip on the set of screw extenders, an open ring clip on the set of screw extenders, a graduated diameter lock on the set of screw extenders, a snap-grip lock on the set of screw extenders, an offset engagement lock on the set of screw extenders. Other types of frictional engagement locks can also be used to secure the extenders together at the underside of the fixation device.

In one example embodiment of the claimed invention, the screw extender for inserting a fixation device into a vertebral bone includes a first extender branch and a second extender branch. The first extender branch includes an elongated portion for extending from a proximal end outside a patient to a distal end and an interlocking tab at the distal end that connects at an undersurface of a screw head. The interlocking tab includes a graduated bilateral locking mechanism to ensure a positive connection to a second extender branch. The second extender branch is substantially parallel to the first extender branch and also includes an elongated portion for extending from a proximal end outside a patient to a distal end. The distal end includes an interlocking tab that connects at the undersurface of the screw head to the first extender branch. The interlocking tab includes a graduated bilateral locking mechanism to ensure a positive connection to the first extender branch. As outlined above, the locking mechanism on the extender branches can include one or more of tab and slot locking mechanisms, spring-loaded clips, open ring clips, graduated diameter frictional locks, snap-grip locks, offset engagement locks, and other types of frictional locks to secure the extender branches at the underside of the fixation device. The extender branches of the screw extenders can be malleable to afford ease of positioning.

### BRIEF DESCRIPTION OF THE DRAWINGS

FIG. 1 is a perspective view of an access needle with removable handle.

FIG. 2 is a schematic illustration demonstrating the insertion of tubular dilators over the access needle implanted in the pedicle of a spinal vertebra of a patient in the prone position.

FIG. 3 is a schematic illustration demonstrating the insertion of the working channel over the tubular dilators and access needle previously implanted in the pedicle.

FIG. 4 is a schematic illustration demonstrating the working channel affixed to a table-mounted flexible arm after removal of the tubular dilators and access needle.

FIG. 5 is a perspective view of the assembly of the screw extenders with interlocking ends beneath a standard pedicle screw head.

FIG. 6 is a schematic illustration demonstrating the standard pedicle screw with screw extenders after insertion into the pedicle through the working channel.

FIG. 7 is a schematic illustration demonstrating the standard pedicle screw with extenders in situ after removal of the working channel.

FIGS. 8A-8B are process flow diagrams for inserting a fixation device in a vertebra in accordance with a method of the claimed invention.

FIG. 9 shows exemplary lock-in and release mechanisms of the screw extenders in accordance with the claimed invention.

### DETAILED DESCRIPTION

One example embodiment of the claimed invention includes a method of inserting a fixation device in a human spine. While examples of the methods refer to the steps in an illustrative method of pedicle screw insertion, the methods, devices, and systems of the claimed invention can also be used for transfacet or other percutaneous screw placement in the spine or elsewhere in the body.

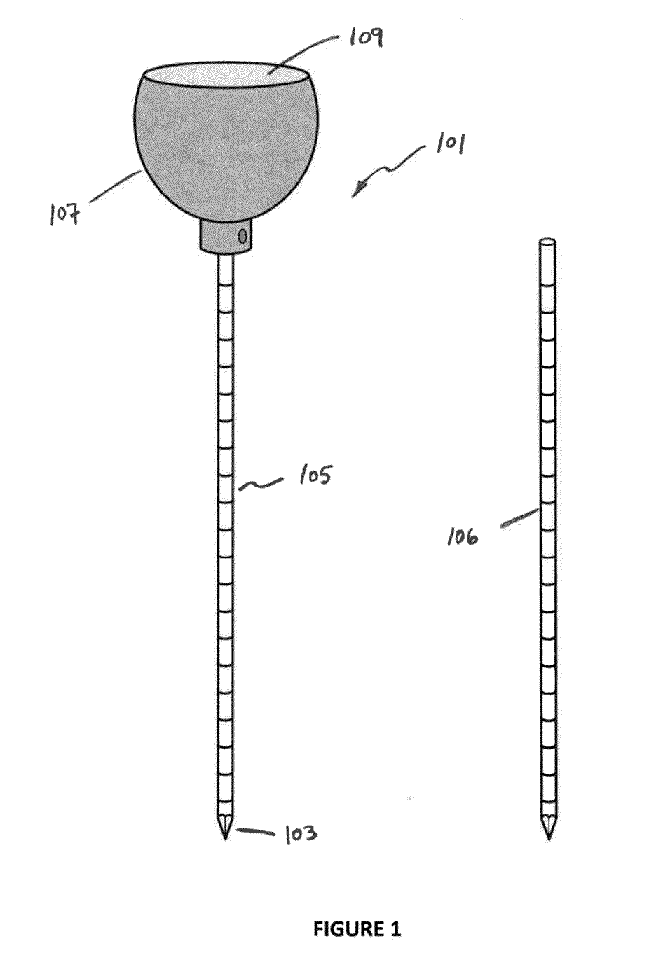

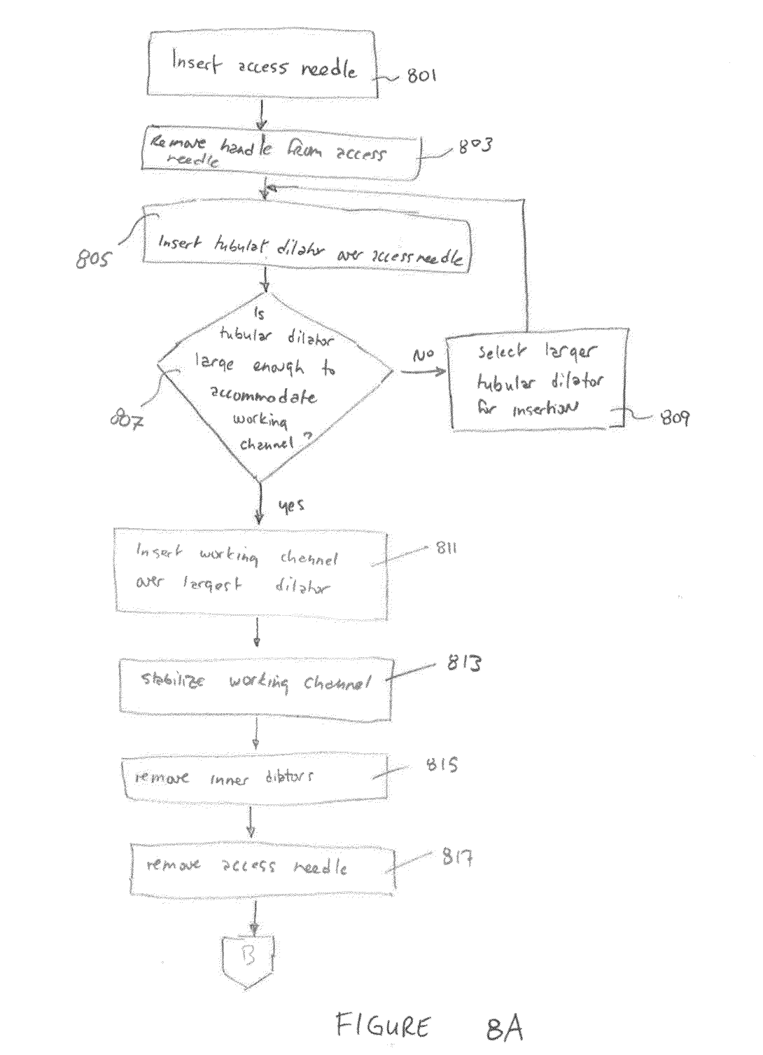

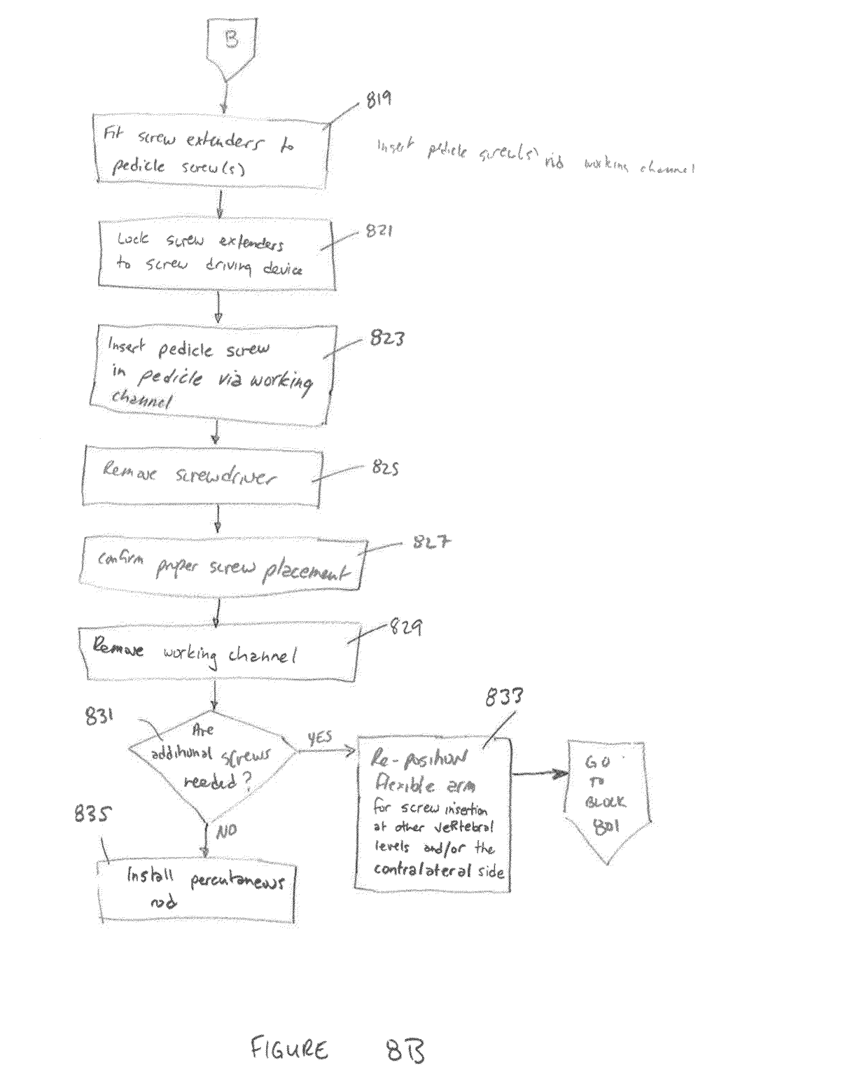

For example, as shown in FIG. 8A, in one example embodiment, the method of inserting a fixation device in a human spine begins by inserting an access needle, such as access needle 101 as shown in FIG. 1. The insertion step is shown in FIG. 8A in block 801.

As shown in FIG. 1, access needle 101 is larger in diameter (for example, by several millimeters) and more robust that a typical Jamshidi needle or other needles with tapered ends (tips). A typical Jamshidi needle can include a hollow outer cannula and an inner obturator with a tapered cutting edge. The access needle of the claimed invention has no inner obturator. For example, access needle 101 can have a diameter of 1-5 mm, with typical diameters of 3-4_mm. Access needle 101 can be a trephine design, which allows for the access needle to bore into a spine or other anatomical structure. The tip 103 is sharp, and the body 105 of the access needle 101 is straight with a width similar to a standard awl or pedicle access instrument (“gearshift”). For example, the width of access needle 101 can be 1-5 mm, with typical widths of 3-4_mm. The handle 107 of the access needle 101 is round (or T-shaped) with a flattened top 109 to allow for tapping with a mallet (not shown). The handle 107 can have a mechanism that allows a locking grip from a radiolucent access needle holder (not shown separately) for positioning with x-ray guidance. For example, the access needle 101 can be inserted with or without an access needle holder into a vertebral body of the spine or other anatomical body using alternating anterior/posterior and lateral fluoroscopy or using other imaging or navigational techniques. The handle 107 of the access needle device 101 is removable. The body of the access needle without the handle (shown in FIG. 1 as reference element 106) can be inserted or removed with a mallet, with a drill, such as a cordless power drill for example, and with other positioning devices as well. As shown in FIG. 8A, in block 803 the handle 107 of the access needle 101 is removed.

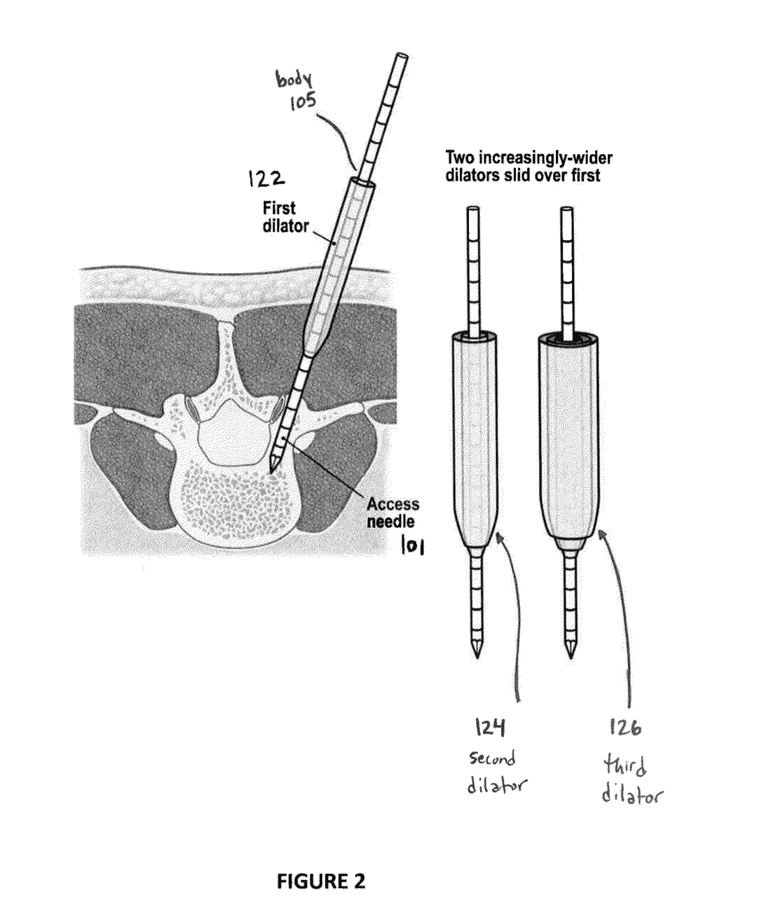

As shown further in FIG. 2, after the access needle 101 is tapped or drilled into the pedicle of a vertebral body using x-ray guidance, the handle 107 of the access needle 101 is removed and a series of tubular dilators are inserted over the body 105 of the access needle 101 down to the junction of the transverse process and facet joint of the vertebrae. That is, the access needle 101 is inserted into the pedicle process that projects dorsally from the superior part of the vertebral body at the junction of the posterior and lateral surfaces. First dilator 122 is inserted over the body 105 of the access needle 101 in block 805 of FIG. 8A. In block 807, the surgeon can evaluate the size of the opening to the spinal anatomy created by the first dilator 122. As shown further in block 809, if the opening to the spinal anatomy needs to be enlarged, the process returns to block 805, and additional dilators 124, 126 can be inserted sequentially, with smaller to larger diameters to progressively increase the size of the surgical opening. In addition to second dilator 124 and third dilator 126, additional dilators can be used to continue to progressively increase the size of the surgical opening. The dilators 122, 124, 126 (and others) are used to gradually separate muscle and other tissue to create an opening large to accommodate surgical tools. Once the opening is large enough to accommodate the surgical tools, the process continues to block 811.

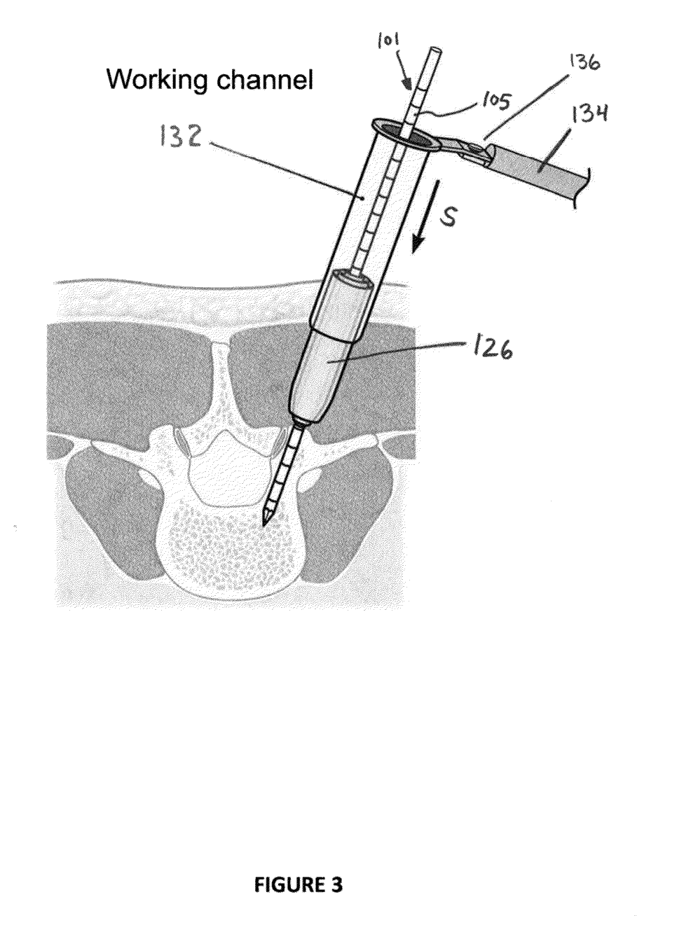

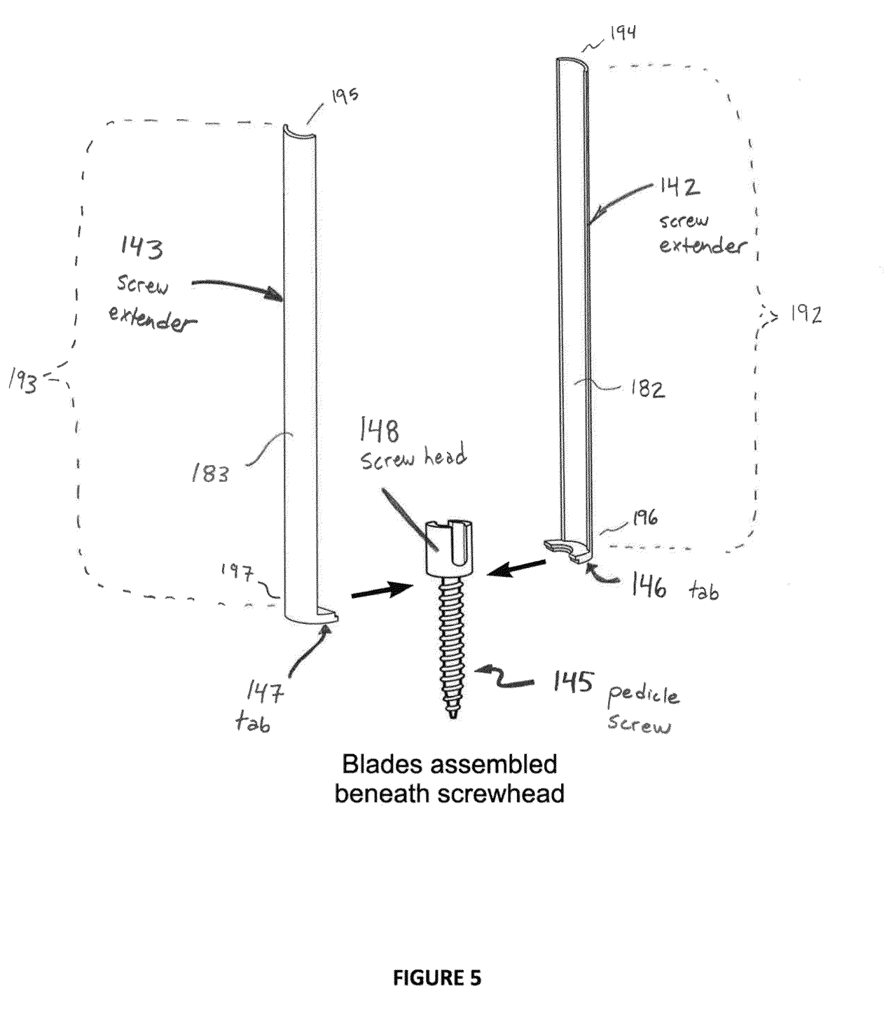

As shown in FIG. 3 and in block 811 of FIG. 8A, when the surgical opening is large enough to accommodate surgical tools such as screw extenders (shown further in FIG. 5), a working channel 132 is slid over the largest dilator, such as dilator 126 as shown in FIG. 3. In sliding the working channel 132 over the largest dilator along direction S, the working channel 132 also slides over smaller diameter dilators and the body 105 of access needle 101. The working channel 132 is then stabilized in position in block 813 of FIG. 8A by securing it to a flexible arm 134 that is attached to an operating table or other secure device (not shown) and fixed in space. The working channel can vary in diameter to accommodate various surgical tools, such as compressors, distractors, rod benders, tipped and untipped probes, persuaders, reducers, grippers, drivers, awls, rockers, positioners, taps, and the like. The arm 134 holds the working channel 132 in place by a frictional connection or by other secure connections. For example, the arm 134 can hold the working channel 132 in place by tightening a clamping mechanism 136 to hold the working channel 132. An adapter (not shown separately) can be fastened to the side rail of the operating table to connect to the flexible arm to provide positioning and performance stability. The adapter can be made long enough to slide the flexible arm up and down the operating table and the patient's spine for multi-level cases and for use on the contralateral side.

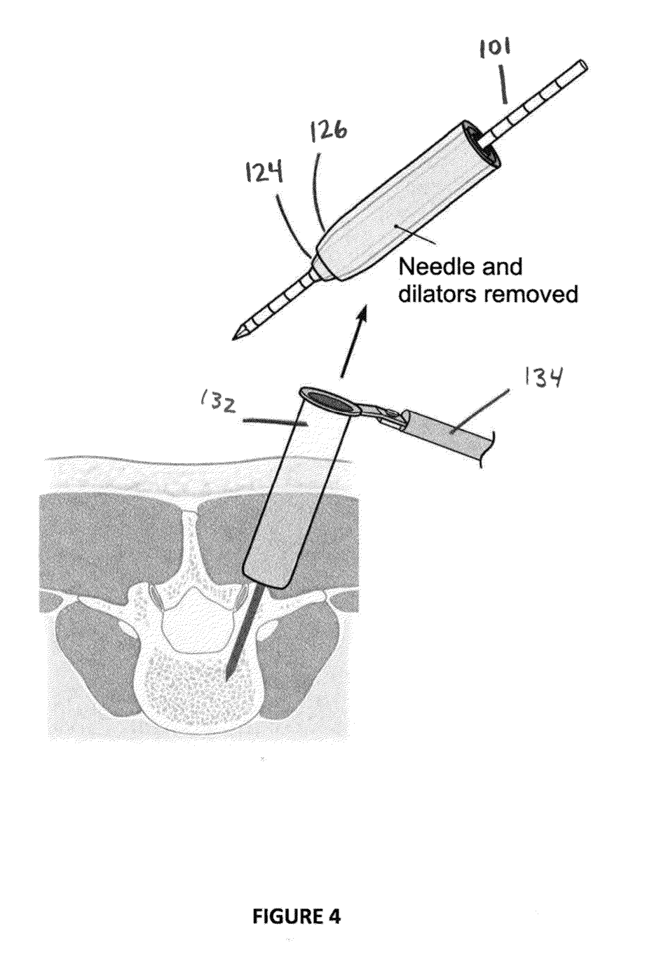

Once the working channel 132 is stabilized, the inner dilators 124, 126 are removed in block 815 of FIG. 8A and as shown in FIG. 4.

After the inner dilators 124, 126 are removed, the access needle 101 can then be removed in block 817. The access needle 101 can be removed manually or with a power drill in reverse or with other positioning aid devices (not shown). Upon removal of the dilators 124, 126 and access needle 101, the working channel 132 affixed to the table-mounted flexible arm 134 serves as the guide for screw insertion into the vertebral bodies. That is, the working channel 132 forms a direct path from outside the patient's body to the vertebra. The distal end of the working channel can have an opening on the lateral side to accommodate the structural presence of the transverse process and ensure a good fit onto the junction of the transverse process and facet joint (not shown).

Once the access needle 101 and the dilators 124, 126 have been removed and the working channel 132 is in place, the process continues in FIG. 8B in block 819, where the pedicle screw(s) will be inserted through the working channel guide. A standard or cannulated percutaneous pedicle screw can be inserted through the working channel with screw extenders.

As outlined above, the system and devices of the claimed invention can be utilized to insert a variety of screws with many types of spinal instrumentation as outlined below. Regardless of the specific screw and instrumentation combination used, probing is used to confirm proper bony margins within the pedicle, and insertion of a tap can be performed following confirmation of the bony margins within the pedicle.

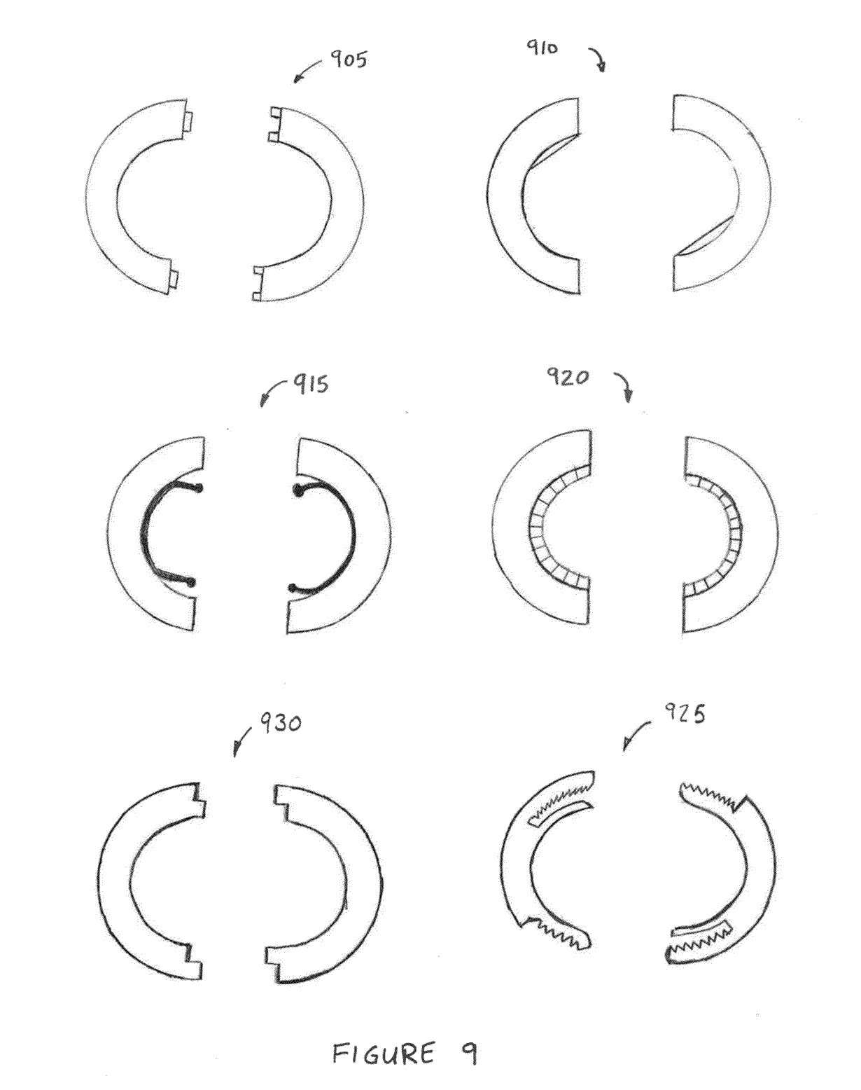

For example, in block 819 paired screw extenders 142, 143 are fitted to a pedicle screw 145 as shown further in FIG. 5. The screw extenders 142, 143 include two interlocking tabs 146, 147 that connect at the undersurface of the screw head 148 bilaterally and lock in. A lock-in and release (unlock) mechanism (not shown separately) is included in the tabs 146, 147 to ensure a positive connection. For example, as shown in FIG. 9, the lock-in and release mechanism can incorporate tab and slot 905 configurations, a spring-loaded clip 910, an open ring clip 915, a graduated diameter lock 920, a snap-grip 925, an offset engagement lock 930, other frictional engagements, and the like. Combinations of these exemplary lock-in and release mechanisms can also be used. The lock-in and release mechanism is graduated to allow for a tight fit or looser fit around the screw head 148 to permit the insertion of instruments around the screw head 148. For example, a tight fit around the screw head is necessary for the insertion of the pedicle screw into bone. The tabs can then be separated in a graduated fashion while still locked under the screw head to allow for the insertion of additional instruments around the screw head to manipulate the screw such as a head turner, reduction device or persuader device.

The screw extenders include a pair of extender branches, such as first extender branch 182 and second extender branch 183. The first extender branch 182 includes an elongated portion 192 extending from a proximal end 194 outside a patient to a distal end 196 and an interlocking tab 146 at the distal end 196 that connects at an undersurface of a screw head (such as that of pedicle screw 145) and includes a graduated bilateral locking mechanism (shown in FIG. 9) to ensure a positive connection to another extender branch (such as second extender branch 183). Second extender branch 183 is substantially parallel to the first extender branch 182 with an elongated portion 193 for extending from a proximal end 195 outside a patient to a distal end 197 and an interlocking tab 147 at the distal end 197 that connects at the undersurface of the screw head and includes a graduated bilateral locking mechanism (shown in FIG. 9) to ensure a positive connection to the first extender branch 182.

The paired screw extenders 142, 143 form a retractor wall on each side of the screw 145 that extends superiorly beyond the skin after screw insertion. The screw extenders 142, 143 can be made of a malleable alloy (or plastic) that is radiolucent. To prevent the attached screw extenders 142, 143 from sliding up and down the body of the screw 145, an additional positioning device (not shown separately) locks the screw extenders 142, 143 to the screwdriver as shown in block 821 of FIG. 8B. The additional positioning device can include a ring with two slits on each end though which tabs 146, 147 are inserted and clamped on the screwdriver. This device prevents the screw extenders 142, 143 from flapping and moving up or down the screwdriver. The screw extenders 142, 143 in accordance with the claimed invention are adaptable to commercially available screws (standard or cannulated).

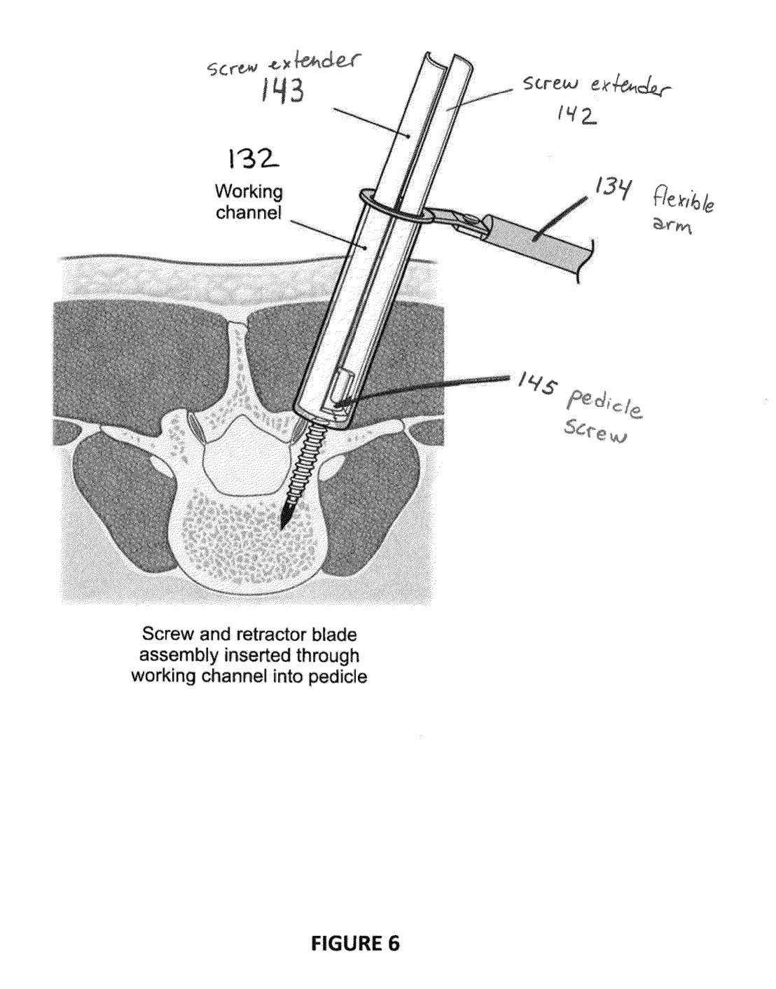

Returning to FIG. 8B, once the screw extenders 142, 143 are locked to the screwdriver, in block 823 a screw, such as pedicle screw 145, is inserted into the pedicle via the working channel 132 with a screwdriver and the screw extenders 142, 143 as illustrated in FIG. 6. Once the screw 145 is inserted into the pedicle, the screwdriver is then removed in block 825 of FIG. 8B, and proper placement of the screw 145 is confirmed using fluoroscopy or other imaging or navigational techniques in block 827.

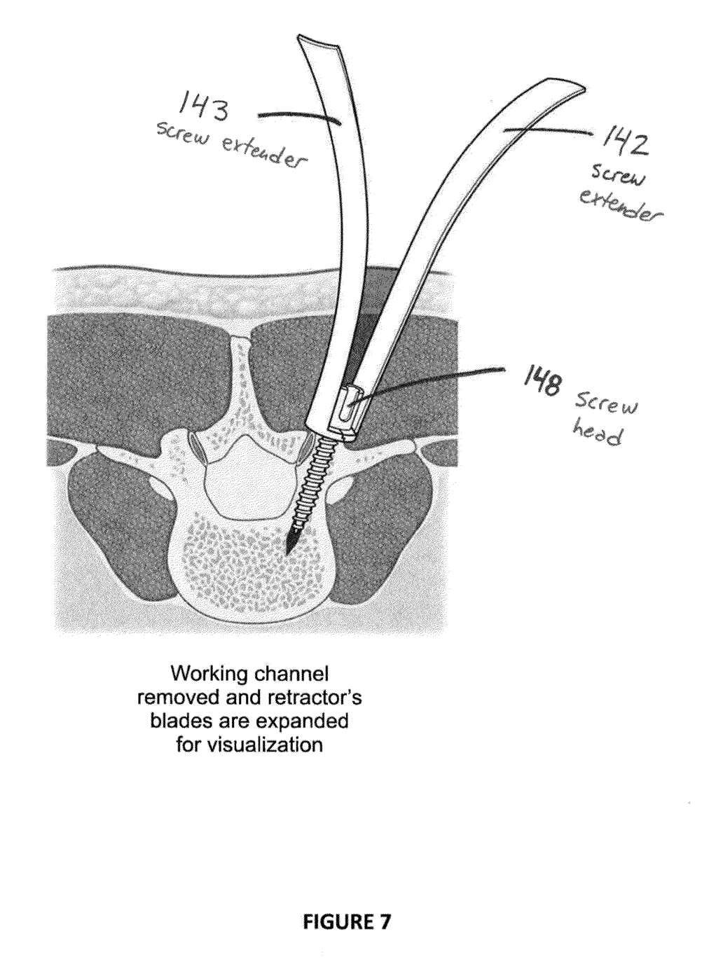

After proper screw placement is confirmed, in block 829 the working channel 132 is disconnected from the flexible arm 134 and removed. FIG. 7 shows the resulting system with the working channel 132 removed and the screw extenders 142, 143 expanded. The screw extenders 142, 143 serve as retractors to allow for stimulation of the screws head 148 for neuromonitoring and percutaneous rod insertion. The malleability of the screw extenders 142, 143 is such that a self-retaining retractor (not shown separately) can be used to open each extender 142, 143 above the skin. Holes can be made in the extenders 142, 143 to allow for retraction with an instrument to facilitate visualization once in place.

The steps outlined above for inserting the pedicle screw are repeated for screw placements on the other (contralateral) side and at other necessary vertebral levels using the devices and system of the claimed invention as shown starting in block 831. For example, in ipsilateral insertions, the same flexible arm 134 is used for the same side placements. The flexible arm 134 can be repositioned at other vertebral levels or spaces by sliding it up or down over the table adapter. For contralateral screw insertions, the same flexible arm can be used or a second flexible arm positioned on the contralateral side of the table can be used. Repositioning of the flexible arm is shown in block 833 in FIG. 8B. The process then returns to block 801 for the additional screw(s).

After all pedicle screws are inserted, in block 835 a rod or other support or alignment device is placed percutaneously. The screw extenders 142, 143 allow for the placement of additional devices for persuasion or reduction that involve gripping the screw head 148. The screw extenders 142, 143 can be opened several notches to facilitate the additional placement. A release mechanism of the screw extenders allows for their removal after the rod is secured to the screws.

Having thus described the basic concept of the invention, it will be rather apparent to those skilled in the art that the foregoing detailed disclosure is intended to be presented by way of example only, and is not limiting. In addition to the embodiments and implementations described above, the invention also relates to the individual components and methods, as well as various combinations and sub-combinations within them. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as can be specified in the claims. Accordingly, the invention is limited only by the following claims and equivalents thereto.

The claimed invention is:

1. A method of securing a fixation device into a vertebral bone, the method comprising:
inserting an access needle with a removable handle in the vertebral bone;
inserting sequentially larger dilators over the access needle;
inserting a working channel over the sequentially larger dilators;
securing the working channel to provide stable access to the vertebral bone;
removing the dilators and access needle;
attaching a set of screw extenders to the fixation device;
inserting the fixation device with the attached set of screw extenders into the working channel; and
securing the fixation device into the vertebral bone through the working channel without the use of a guide wire.

2. The method of securing a fixation device into a vertebral bone of claim 1, wherein the fixation device is a pedicle screw.

3. The method of securing a fixation device into a vertebral bone of claim 1 further comprising:
securing a plurality of fixation devices in at least one vertebral bone.

4. The method of securing a fixation device into a vertebral bone of claim 3 further comprising:
installing a support or alignment rod to the plurality of fixation devices.

5. The method of securing a fixation device into a vertebral bone of claim 1, wherein attaching the screw extender to the fixation device includes connecting interlocking tabs on the screw extender to an undersurface of the fixation device.

6. The method of securing a fixation device into a vertebral bone of claim 1, further comprising:
locking the set of screw extenders to the fixation device.

7. The method of securing a fixation device into a vertebral bone of claim 6, wherein locking the set of screw extenders to the fixation device includes connecting a tab and slot locking mechanism on the screw extender.

8. The method of securing a fixation device into a vertebral bone of claim 6, wherein locking the set of screw extenders to the fixation device includes engaging a spring-loaded clip on the set of screw extenders.

9. The method of securing a fixation device into a vertebral bone of claim 6, wherein locking the set of screw extenders to the fixation device includes engaging an open ring clip on the set of screw extenders.

10. The method of securing a fixation device into a vertebral bone of claim 6, wherein locking the set of screw extenders to the fixation device includes connecting a graduated diameter lock on the set of screw extenders.

11. The method of securing a fixation device into a vertebral bone of claim 6, wherein locking the set of screw extenders to the fixation device includes connecting a snap-grip lock on the set of screw extenders.

12. The method of securing a fixation device into a vertebral bone of claim 6, wherein locking the set of screw extenders to the fixation device includes connecting an offset engagement lock on the set of screw extenders.

13. A screw extender for inserting a fixation device into a vertebral bone, the screw extender comprising:
a first extender branch with an elongated portion for extending from a proximal end outside a patient to a distal end and an interlocking tab at the distal end that connects at an undersurface of a screw head and includes a graduated bilateral locking mechanism to ensure a positive connection to another extender branch; and
a second extender branch substantially parallel to the first extending branch with an elongated portion for extending from a proximal end outside a patient to a distal end and an interlocking tab at the distal end that connects at the undersurface of the screw head and includes a graduated bilateral locking mechanism to ensure a positive connection to the first extender branch.

14. The screw extender for inserting a fixation device into a vertebral bone of claim 13, wherein at least one of the first extender branch and the second extender branch is malleable.

15. The screw extender for inserting a fixation device into a vertebral bone of claim 13, wherein the bilateral locking mechanism includes a tab and slot locking mechanism on the set of extender branches.

16. The screw extender for inserting a fixation device into a vertebral bone of claim 13, wherein the bilateral locking mechanism includes a spring-loaded clip on the set of extender branches.

17. The screw extender for inserting a fixation device into a vertebral bone of claim 13, wherein the bilateral locking mechanism includes an open ring clip on the set of extender branches.

18. The screw extender for inserting a fixation device into a vertebral bone of claim 13, wherein the bilateral locking mechanism includes a graduated diameter lock on the set of extender branches.

19. The screw extender for inserting a fixation device into a vertebral bone of claim 13, wherein the bilateral locking mechanism includes a snap-grip lock on the set of extender branches.

20. The screw extender for inserting a fixation device into a vertebral bone of claim 13, wherein the bilateral locking mechanism includes an offset engagement lock on the set of extender branches.
